(19) United States
(12) Patent Application Publication
(10) Pub. No.: US 2004/0210236 A1
(43) Pub. Date: Oct. 21, 2004

(54) Flow path control catheter with funnel-shaped expandable structure at proximal part and tubular-shaped structure at distal part and perfusion system with such a catheter

(76) Inventors: Mats Allers, Lund (SE); Krasnodar Ivancev, Lund (SE); Bengt Jeppsson, Lund (SE); Anders Lunderquist, Lund (SE); Leo Schultze Kool, Nieuw Vennep (NL)

Correspondence Address: LADAS & PARRY, 224 SOUTH MICHIGAN AVENUE, SUITE 1200, CHICAGO, IL, 60604, US

(21) Appl. No.: 10/483,034

(22) PCT Filed: Jul. 8, 2002
(86) PCT No.: PCT/SE02/01360

(30) Foreign Application Priority Data
Jul. 9, 2001 (SE) 0102446-2
Jul. 9, 2001 (SE) 0102448-8

(51) Int. Cl. A61F 11/00

(52) U.S. Cl. 606/108; 604/1

(57) ABSTRACT

The present invention relates to a catheter (100), comprisig a catheter stem (104) and a radially expandable structure (102), said expandable structure being configured to adopt in an expanded state a funnel like shape at a first proximal part that is attached to an end of said catheter stem (104) and an elongate substantially tubular shape at a second distal part. A selected portion of the expandable structure is coated with an unpermeable material to realise an occluding seal. The invention also relates to a catheter set, a perfusion system and a device for controlling the flow path in the inferior Venn cava.

### TECHNICAL FIELD

The present invention relates in general to a catheter and a catheter set devised for controlling the flow path in a blood vessel. More specifically, the invention is as an example applied in controlling the blood flow in vena cava inferior in a liver perfusion system and in a method for non-surgical perfusion of a liver in a living being.

### BACKGROUND OF THE INVENTION

Treatment with systemic chemotherapy is one of the presently used possibilities for cancer treatment. However, substances that are effective in this kind of treatment are often harmful to the system of the body as a whole. Particularly, the treatment of cancer of the liver presents a serious clinical problem, and the success rate when treating liver cancer is today very low.

Although primary liver cancer (hepatoma) is rather uncommon in northern Europe and United States, hepatoma is prevalent in other parts of the world, e.g. in Southeast Asia, Japan, the Pacific Islands, Greece, Italy and parts of Africa. Also, many patients with cancer in the gastrointestinal tract develop isolated hepatic metastases, since the liver is the primary target for dissemination. Due to the distribution of the metastases within the liver, only few patients with liver cancer can be cured by resection.

Liver cancer is today mainly treated with systemic chemotherapy. However, no substantial increase in the time of survival of the patients is following this treatment (L. M. De Brauw “Isolated liver perfusion. An experimental modality in the treatment of hepatic metastases.” Thesis, University of Leiden, Leiden, The Netherlands.). A reason for these discouraging results seems to be the fact that the toxicity of the chemodrugs limits the possible dosage due to the systemic effects. Local administration by infusion in the hepatic artery does not solve this problem, since the chemodrugs are distributed in the system also during this procedure.

Therefor, it has been suggested that therapeutic drugs should be administrated locally by performing perfusion of the liver or other selected isolated organs.

1. State of the Art

Examples of techniques for isolating and perfusing organs are found for example in the following prior art documents.

In EP-0 364 799 to BGH Medical Productions, a process of perfusing a high concentration of an agent through an organ is described. The agent is infused arterially in the organ and on the venous side of the organ the blood is removed from the body using a specially designed double balloon catheter. In this process there is a leakage to the systemic blood flow, since there are numerous blood communicating vessels besides the main artery and the main vein.

A similar catheter is used in the U.S. Pat. No. 5,817,046 to Glickman et al., which is hereby incorporated by reference, showing a system for perfusion of the pelvic cavity. The pelvic cavity is isolated between a double catheter, placed in the iliac vein, and bilateral thigh tourniquets. The thigh tourniquets, which are used to restrict the flow of blood between the legs and the pelvic cavity of the patient, limit the time during which perfusion can be performed.

In U.S. Pat. No. 4,714,460 to Calderon, which is hereby incorporated by reference, feedback methods and systems for retrograde perfusion in the body are described. A double balloon concentric catheter, with an inner infusion lumen and an outer suction lumen, is used for perfusion of the venous side of the vascular network. The therapeutic agent for treatment is infused inside the vein in the opposite direction with respect to the ordinary blood flow, also called retrograde infusion. The described method is, thus, designed to operate in back pressure and the perfusion fluid is continuously diluted by arterial blood.

U.S. Pat. No. 4,883,459 to Calderon, which is hereby incorporated by reference, describes a method for perfusion where a carrier medium dye is injected into the tumour. The flow of the dye is monitored to determine an optimal retrograde perfusion path through the tumour.

A balloon catheter with closed tip and device for perfusion with such catheters, are described in U.S. Pat. No. 5,746,717 to Aigner, which is hereby incorporated by reference. The catheter has at least one contrast marking which enables the position of the catheter inside the body to be determined.

The perfusion processes and apparatuses described above all include the return of the blood, which has been contaminated with drugs, to the systemic circulation. This requires treatment to remove the contaminants before this blood can be returned to the body. Furthermore, the catheters mentioned above all have the disadvantage that they remain in the body of the living being after termination of the perfusion process.

An assembly for hepatic isolation and perfusion is described in U.S. Pat. No. 4,192,302 to Boddie, which is hereby incorporated by reference. This assembly allows the blood from the intestines and the lower parts in the patient's body to flow unimpeded through a plurality of shunts. Meanwhile, the blood in the isolated liver is circulated using a heart-lung machine, which allows cancericidal doses of drugs to be delivered to liver cancers essentially without systemic effects. However, the procedure involved is complicated and the large operation, which is needed to place the shunts, only permits perfusion once inter alia due to scars in the tissues and the severe stress on the body of the patient. Consequently, a drawback with some of the above mentioned, earlier procedures for organ perfusion is that the organ may not be isolated in a perfusion circuit, thus, perfusion fluid may easily leak into the systemic circulation. Another drawback is that blood, which is used to perfuse the organ, may after perfusion contain therapeutic agents, and thus, needs to be purified before it is returned to the body. In the case of a surgical method, as the one described in U.S. Pat. No. 4,192,302, it is a disadvantage that the perfusion can only be performed once on each patient due to the large operation involved.

In WO 9933407 to Heartport a schematically described system for non-surgical isolation and perfusion of organs, inter alia in a liver is found. The document WO01/03755 to Jostra shows a more specific method and catheter set for isolating a liver while simultaneously managing the systemic blood flow between the upper and the lower body parts.

2. Problems in Prior Art

A problem in some of the prior art occurs when there is a considerable fraction of the blood flow that does not enter or leave the organ through the main input and output blood vessels, which for example is the case in the liver. There is thus a risk for leakage of perfusion fluid through these minor vessels to the systemic circulation.

In connection with for example the isolation of a liver or for other purposes there is a problem of controlling the blood flow in the area where the hepatic veins opens into the inferior vena cava. Prior art suggests balloon catheters to occlude the inferior vena cava. However, experimental tests show that the balloons are difficult to position.

A further problem is to achieve a satisfactory sealing against leakage past an occlusion in the inferior vena cava. The inferior vena cava is a comparatively large vein with resilient walls that easily expand when a balloon is dilated inside it.

Another problem is to achieve a selectable flow path or occlusion within the inferior vena cava and the veins that join vena cava inferior.

### OBJECT OF THE INVENTION

The general object of the present invention is to provide a catheter that solves the problem of controlling the flow path for e.g. blood in a blood vessel.

Aspects of the problem are:

to achieve a safe positioning of the catheter in a blood vessel;

to achieve a satisfactory sealing in the occlusion of a blood vessel; and

to achieve a selectable flow path or occlusion in a blood vessel having adjoining or branching vessels.

A more specific problem is to provide a catheter that enables controlling the blood flow in the inferior vena cava.

### BRIEF DISCLOSURE OF THE INVENTION

The object of the invention is achieved by means of a catheter having at its distal end a member with a radially expandable structure, for example in the shape of a mesh structure adopting the function of a stent. In an expanded condition this structure takes on the form of an elongated umbrella or an elongated bell, that positioned in a blood vessel presses its outer periphery against the inner wall of the vessel and thereby contributes to the positioning of the catheter.

In order to achieve a selectable flow path in the vessel a selectable part of the structure is covered with an unpermeable coating, whereas the uncoated part of the structure is permeable to a fluid such as blood or a perfusion liquid. In use the coated part and the uncoated parts of the structure are positioned in the vessel and in relation to vessel branches such that a selected flow path is enabled or a selected flow path is occluded dependent on the specific application. Further control or selection of flow path is achieved by selectively using a lumen in the catheter to lead a liquid to or from the inside of the expandable structure.

The sealing of a flow path is further improved by the fact that the coating is contributory pressed in the radial direction against the inner wall of the blood vessel and in some applications against the orifice of a branching vessel by the flow inside the structure. In some applications this effect can be further enhanced by seeing to that the (hydraulic) pressure inside the coated structure is higher than the pressure on the outside.

For the purpose of accurately positioning the expandable structure embodiments of the invention comprises a combination of two similar or two different varieties of the inventive catheter structure. In practical use, these two structures are inserted into a desired location in a blood vessel from opposite directions until the respective distal ends of the two catheters are joined. In this condition the position is controlled from two directions and the structures are expanded. In order to secure the catheters further in their position, the distal ends of the respective structures are interlocked. In one embodiment, the inventive catheter is therefore provided with an interlocking mechanism at the distal end, also known as a stent graft. In another embodiment, the inventive catheters are configured such that the expandable structure of a second catheter is expanded inside the already expanded structure of a first catheter. Thereby in particular the first catheter is additionally locked in its position. This latter interlocking mechanism is also known as a stent graft.

A particularly tricky and at the same time illustrative application for the invention is presented by the control of the flow in the inferior vena cava (or vena cava inferior). The invention is therefore explained below by means of an example taken from this application. The control of the blood flow in vena cava inferior also present more specific problem aspects that are solved by the invention.

Such a specific aspect of the present invention is to provide a catheter that substantially reduces or eliminates the outflow from the hepatic veins into the inferior vena cava and thereby minimises the possibility of releasing a therapeutic compound from an isolated and perfused liver into the systemic circulation.

Another specific aspect is to close the blood flow from the inferior vena cava into the right atrium of the heart. Selectively, this aspect is combined with the aspect of maintaining a flow path through the orifice of the hepatic veins from the inferior vena cava.

The catheter is removable from the intravascular part of the living being without harm to the living being, such as after treatment of a liver using a non-invasive liver perfusion system. The invention thus also relates to a liver perfusion system and a method, wherein the catheter is used in medical treatment.

The invention also relates to a liver perfusion system and a method for liver perfusion, in which system and method the catheter is used.

### BRIEF DESCRIPTION OF THE DRAWINGS

The invention is further explained with reference to the enclosed drawings in which:

FIG. 1 shows schematically a catheter positioned in the inferior vena cava;

FIG. 2 shows schematically a liver connected to a liver perfusion system comprising the catheter of FIG. 1;

FIG. 3 shows schematically an embodiment of the inventive catheter in combination with a balloon catheter;

FIG. 4a-4b show schematically different combinations of the inventive catheter applied to interlock in a selected position in a blood vessel;

FIG. 4c shows schematically an embodiment of the inventive catheter combination applied in a liver perfusion system;

FIG. 5a-5f show different embodiments of the inventive catheter; and

FIG. 6 shows another embodiment of the inventive catheter.

### DETAILED DESCRIPTION OF THE INVENTION

The present invention thus relates to a catheter for use in controlling the flow path in a blood vessel. For the sake of explanation the invention is described below by means of an exemplifying application in a liver perfusion system, where the inventive catheter is applied in controlling the flow path in the inferior vena cava in the area where the hepatic veins joins the inferior vena cava.

In this embodiment the inventive catheter has the function and effect of substantially reducing or even eliminating leakage of perfusion fluid from the liver through the hepatic veins into the inferior vena cava, and thereby into the systemic circulation of the organism. Leakage of fluids from the hepatic veins into the inferior vena cava is dependent on the amount and pressure of the perfusion fluid used in the perfusion system and the leakage is difficult to eliminate completely. Therefor a leakage of about 1-2% of the perfusion fluid may be an amount that has to be accepted. A careful control of the pressure and the leakage is highly important to reduce the possibility of large leakage problems into the inferior vena cava, which would increase the contamination of the systemic circulation. An increase in pressure and amount of perfusion fluid may result in an increase in the leakage and thereby an increase of the amount of perfusion fluid entering into and contaminating the systemic circulation. A proper use of the inventive catheter would reduce or eliminate these problems.

The distal end of a catheter is in this text intended to mean the end that is intended to be inserted into the body whereas the proximal end is the end remaining close to the operator of the catheter.

Furthermore the inventive catheter is removable after use. In treatments involving the invention, there is in most cases a need to reinstall the natural flow paths of the organism. So, as for example in the performance of minimally invasive liver perfusion, it is highly important to be able to substantially isolate the liver from the systemic circulation inter alia by occluding the hepatic veins. Likewise, the occlusion must be reversible without damaging any blood vessel tissue.

Catheter Embodiments

The invention relates to a catheter configured and dimensioned to be introduced into a blood vessel such as an artery or a vein. In the exemplifying embodiment, the catheter is configured and dimensioned to be introduced into the inferior vena cava either from above via the atrium or from below via for example a femoral vein. The catheter is preferably adapted to be introduced by means of the per se known seldinger technique. Therefore such embodiments are provided with and adapted to be operated by means of a guide wire and an introducer. The inventive catheter thus comprises an elongate member with a member having an expandable structure at its distal end and a mechanism for operating expansion and deflation, for example by means of a wire leading through the elongate member or by means of an introducer sheath. Different embodiments comprise one or more lumen in order to provide a flow path connected to the inside of the expandable structure.

When introduced into the blood vessel and positioned at the desired location, for example in the inferior vena cava in the area of the hepatic veins, the expandable member of the catheter is expanded to a larger perimeter or diameter. The outer mantle of the expandable member fixates the catheter in the selected position in the vessel by pressing against the inner wall of the vessel. In one variety of the invention at least a part of the expandable structure is coated with a material that is devised to seal off the flow path when it is pressed against the inner wall of the vessel during expansion. In another variety of the invention at least a part of the expandable structure is permeable for a fluid to flow through the expandable structure. The expandable structure is for example made in a mesh material, possibly being preloaded such that it tends to expand by virtue of spring forces when released from an introducer sheath. Conversely, the diameter of the structure is reduced when retracted into the introducer sheath. The coating is for example made in a suitable biocompatible material such as silicone rubber.

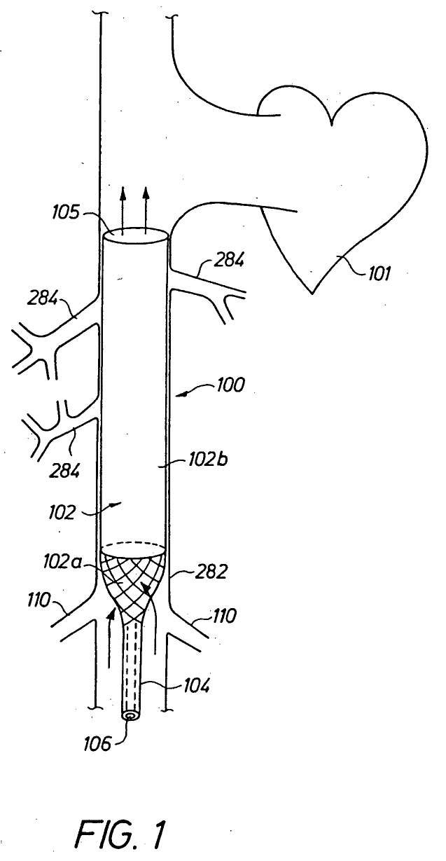

FIG. 1 shows schematically an embodiment of the present invention applied for controlling the flow paths in the inferior vena cava 282, and more specifically in the area of the hepatic veins 284 below the right atrium of the heart 101. A catheter 100 is configured and dimensioned to be placed in the inferior vena cava 282. The catheter 100 comprises at the distal end of an elongate part 104, hereinafter called catheter stem, an expandable structure 102 making up a stent. The expandable stent comprises a permeable part 102a close to the stem and an unpermeable sealing part 102b towards the distal end. The sealing part 102b constitutes a distal expandable occlusive seal that in its expanded form takes on an elongated tubular shape with an open end portion 105. In the shown position the sealing part extends over the hepatic veins 284 up to the part of the inferior vena cava that enters the right atrium. The proximal permeable part 102a takes in its transition from the catheter stem on a bell like or a funnel like shape that in this embodiment is made of a mesh or mesh like structure that preferably extends towards the distal end of the expandable part 102. The envelope surface of the sealing part 102b, which preferably is a part of the mentioned mesh structure, is coated with a sealing film that is pressed against the inner wall of the inferior vena cava. In the shown position, the sealing part of the expandable structure covers and seals off the orifices of the hepatic veins 284. At the same time there is a maintained flow path (shown by filled arrows) through the permeable mesh part 102a, inside the tubular sealed part and through the distal open portion 105. The sealing effect can be further improved by achieving a lower pressure in the hepatic veins than in the inferior vena cava, as the case may be when perfusing an isolated liver. In specific applications the stent can be positioned such that the sealing part 102b covers venae lumbalis 110 where they join the inferior vena cava. In one embodiment the stent is configured and dimensioned such that the sealing part 102b extends over venae lumbalis 110 as well as venae hepatica 284.

In embodiments realising the expandable structure by means of a mesh, the way the mesh is produced, i.e. the spinning, influence the possibility of the mesh to expand into the desired size. In order to realise the unpermeable, sealing part of the stent the mesh is preferably covered by a film for example made in PTFE (polytetrafluorethylene). Other suitable materials that are not toxic, allergenic or cause any other harm to the living being and at the same time is not permeable for body fluids are conceivable.

The elongate catheter stem 104 comprises in different embodiments one or more lumen 106 in order to infuse a fluid into the flow path through the inside of the stent or to establish a flow path through a lumen out from the inside of the stent. As mentioned above, the catheter stem 104 preferably also comprises a lumen for a guide wire and fits into an introducer sheath.

The catheter is in use inserted into the blood vessel, in this example the inferior vena cava by means of the introducer, and is delivered in the inferior vena cava up towards the right chamber of the heart. The introducer is then retracted while the catheter is kept in place by means of the catheter stem 104. As the introducer is removed, the expandable stent structure 102 is released from the inside of the introducer into the vessel and is expanded to a size that is limited by the inner walls of the blood vessel. The flow rate by which the blood flow passes through the stent 102 and further out into the systemic circulation, is substantially the same as prior the insertion of the catheter.

Liver Perfusion System

Although the invention can be used to control the flow paths in different blood vessels it is in particular applicable when performing perfusion of an essentially isolated organ in order to perfuse the organ with a perfusion fluid for example containing a therapeutic agent such as cytostatic drugs. The perfusion can be performed in antegrade or in retrograde. Antegrade perfusion means that the flow of the perfusion fluid in the perfused organ is of the same direction as the normal systemic blood flow and retrograde means that the perfusion flows are redirected compared to the normal blood flow in the perfused organ. In the selection and maintaining antegrade or retrograde perfusion as well as in order to avoid substances to enter the systemic blood flow there is the need for controlling the flow.

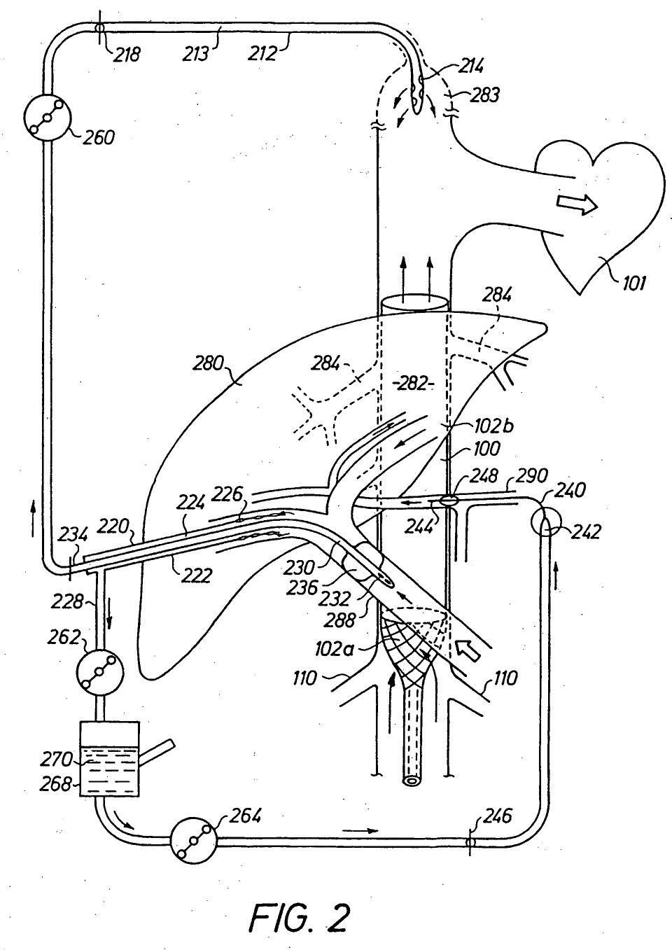

FIG. 2 shows schematically the inventive catheter applied in an embodiment of a liver perfusion system. The liver perfusion in FIG. 2 is carried out in antegrade/retrograde on an isolated liver 280 of a human being with inflow through the hepatic artery and outflow through the portal vein and the hepatic veins blocked. The flow directions in the catheters are indicated by thin arrows, and the normal directions of the systemic blood flow are indicated by thick arrows.

A first catheter 100 as described above in connection with FIG. 1 is placed with its partly permeable stent downstream the heart with the unpermeable sealing part 102b blocking the flow of fluid from the hepatic veins into the inferior vena cava.

A second catheter 212, having a bypass lumen 213 is placed in superior vena cava via the venajugularis of the patient.

A third catheter 220 is placed in vena porta 288. The third catheter 220 is devised to be placed in vena porta percutaneously and trans-hepatically by means of an introducer with a maximum outer diameter of about 12 French. The third catheter 220 is provided with an introducer 222, having an introducer lumen 224 with distal 226 and proximal 228 end openings and being devised to operate as a perfusion lumen. The third catheter 220 further comprises a bypass lumen 230 with distal 232 and proximal 234 end openings and an occlusive seal 236. The third catheter is devised such that the occlusive seal is positioned between said distal end opening of the bypass lumen and said distal end opening of the introducer lumen during operation of the system. Vena porta 288 branches into several veins before it enters the liver and the seal 236 of said third catheter 220 is therefor placed upstream these branches. The vena porta is difficult to enter due to its position between the liver and the intestinal parts and vena porta is therefore entered through the liver, which due to its structure remains relatively undamaged.

A fourth catheter 240 is placed in the hepatic artery 290. The fourth catheter is provided with a perfusion inlet lumen 242 with distal 244 and proximal 246 end openings and an occlusive seal 248. Said distal end opening is positioned distally of the occlusive seal. The fourth catheter 240 is devised to be placed in the hepatic artery 290 by means of an introducer with a maximum outer diameter of 5 French. The occlusive seal of said fourth catheter is capable to efficiently seal off the hepatic artery.

A partially extra-corporeal bypass circuit is formed by connecting the proximal end opening 234 of the bypass lumen of said third catheter to a bypass pump 260. The bypass pump 260, e.g. a roller pump, is further coupled to the proximal end opening 218 of the bypass lumen of the second catheter 212. In the bypass circuit the venous blood from the intestines is taken out through the lumen of the third catheter, re-entered into the systemic blood circulation above the liver 280 through the second catheter 212.

Another partially extra-corporeal perfusion circuit is formed by connecting the proximal end opening 228 of the introducer lumen of said introducer 222 of said third catheter with a perfusion fluid reservoir 268 via means 262 for establishing a negative relative pressure in the liver, e.g. a pump such as a roller pump. The proximal end opening 228 of the lumen of the third catheter 220 and the proximal end opening 246 of the lumen of the fourth catheter are connected to the reservoir 268 via a pump 264, e.g. a roller pump. The perfusion fluid is pumped from the reservoir into the hepatic artery 290, and to the reservoir from vena porta. The portal vein (vena porta) is advantageous to use for establishing a negative relative pressure at the perfusion outlet of the liver, since it is surrounded and strengthened by a relatively rigid structure that prevents the portal vein from collapsing. In embodiments of the invention when this feature does not occur in the vessels entering or leaving the organ to be perfused, it is conceivable to provide, at the perfusion outlet opening of a catheter, a structure devised to prevent the vessel from collapsing, for example in the form of a stent. An increase in pressure and amount of perfusion fluid result in an increase in the leakage and thereby an increase of the amount of perfusion fluid entering into and contaminating the systemic circulation.

The extra-corporeal parts of the bypass and perfusion circuits consist mainly of tubing, which is connected to the proximal ends of the catheters through per se known connections. In this embodiment these connections are e.g. luer lock connections, but other types of connections such as screw connections are conceivable.

By sealing off the flow of blood as described above the liver is essentially isolated from the systemic circulation. Although, there is still a number of minor arteries and veins which still connect the organ to the rest of the body. Before starting the circulation in the bypass and the perfusion circuits, the circuits are filled with fluid in order to ensure that air does not enter the body through the circuits. Circulating the blood past the liver in the bypass circuit is achieved by pumping. The pressure of the venous blood in the lower parts of the body and in vena porta is not high enough to press the blood all the way to the outlet of the perfusion circuit. It is, however, critical that the normal blood pressure upstream the seal 236, of the third catheter in vena porta is maintained and without the bypass circuit the pressure would build up in vena porta. Such a pressure increase could cause severe problems due to open arteries in the flow to the lower part of the body.

Therapeutic agents to be used for treatment of the perfused liver is entered into the perfusion circulation through the perfusion fluid reservoir or by infusing into a branch of an inlet catheter. For this purpose the fourth catheter 240 is suitable, and a therapeutic agent can be infused into the hepatic artery 290.

Further Catheter Embodiments

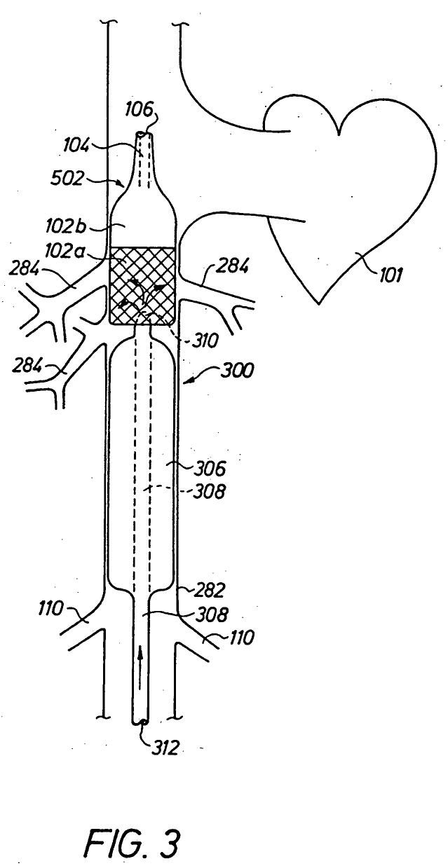

FIG. 3 shows schematically another embodiment of the inventive catheter in a catheter combination 300 with a balloon catheter 306 configured for and positioned in the inferior vena cava. In this embodiment of the expandable stent member 502 the unpermeable part 102b is positioned in the part closest to the catheter stem 104 whereas the permeable part 102a is positioned towards the distal end of the catheter. In the shown position the partly permeable and partly unpermeable stent has been introduced into the vena cava inferior from above via the right atrium of the heart. The permeable mesh 102a is positioned at the orifice of the hepatic veins 284 and thereby allows a flow path through them. The unpermeable sealing part 102b seals off the flow path of the inferior vena cava into the right atrium. Below the expandable stent a catheter with an occlusive balloon is positioned to seal off the flow path in the inferior vena cava 282 below the hepatic veins 284.

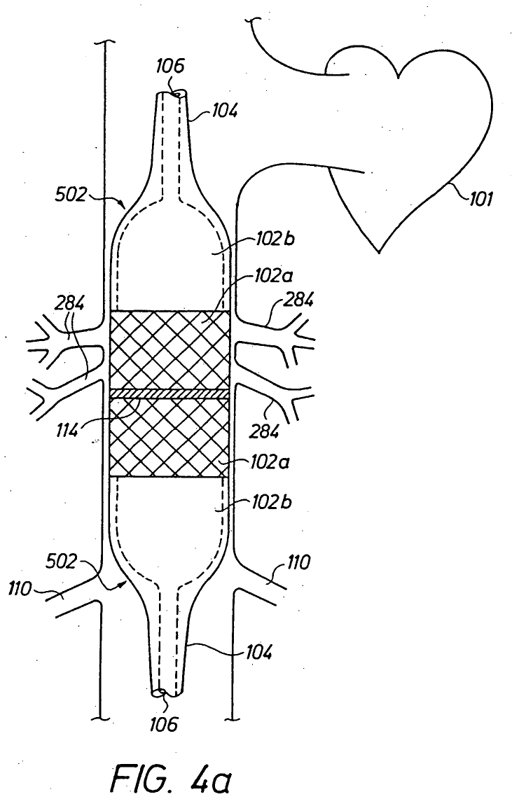

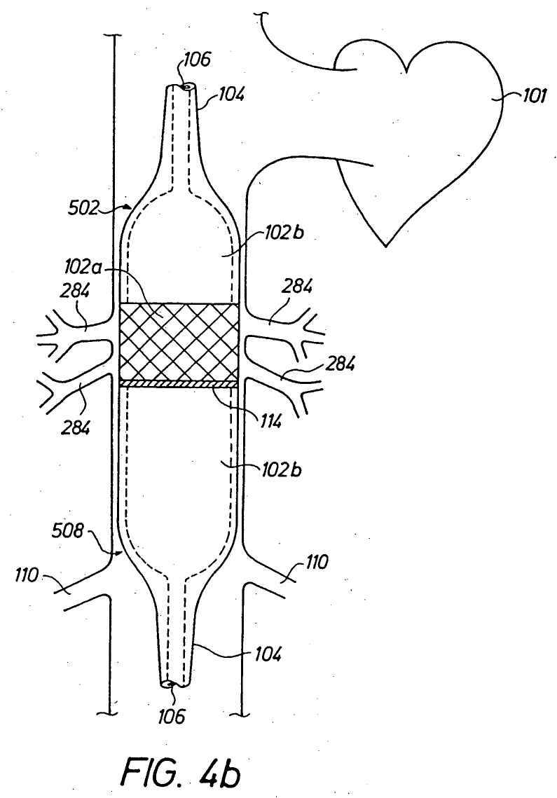

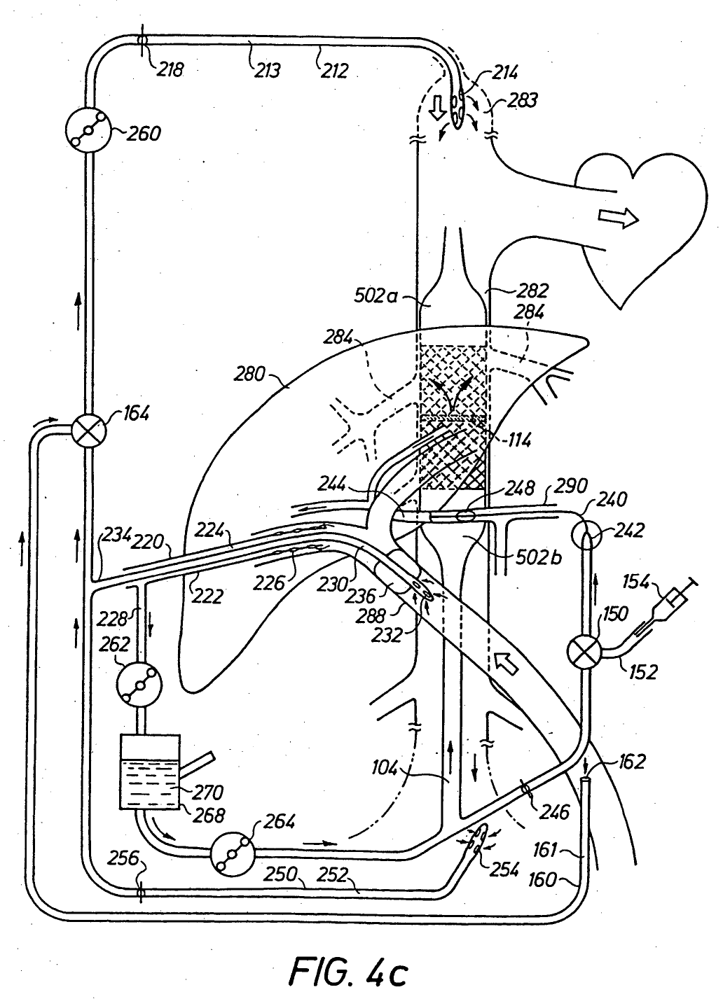

FIG. 4a shows schematically a combination of the inventive catheter stent shown in FIG. 3. Thus, in this configuration two similar or identical partly coated stents are positioned in the blood vessel which in the example of FIG. 4a is the inferior vena cava just below the right atrium. The two stents have been inserted to their respective positions from opposite directions. One of the stents is first expanded to press against the inner wall of the blood vessel thus forming a tubular space. Thereafter the second stent is positioned such that it at its distal end extends somewhat into the tubular space inside the expanded first stent. The second stent is expanded to press against the inner wall or wall structure of the first catheter with a part of its mantle surface and against the inner wall of the blood vessel with the remainder of the expanded structure. Thereby the two stents are mutually interlocked in an interlocking section 114 to further secure the selected or desired position in the vessel. This functionality is also known as a stent graft. The positioning security is thus improved by the mutual friction achieved between the expanded stent parts and the expansion pressure against the inner wall of the vessel on one hand. On the other hand by the capability of being adjusted by holding, pulling or pushing the catheter stem from the outside of the body.

The positioning problem is specifically accentuated in the inferior vena cava in situations when the flow path into the atrium should be occluded in the short distance between the inlet area of the hepatic veins and the atrium. In the embodiment of FIG. 4a, the coated unpermeable part 102b of the upper stent is positioned to occlude this area, whereas the permeable part 102a is positioned with its mesh structure over the inlet openings of the hepatic veins. Conversely, the coated unpermeable part 102b of the lower stent is positioned to occlude the inferior vena cava from the inlet opening of the hepatic veins to the renal veins, whereas the permeable part 102a is positioned with its permeable part over or in the vicinity of the outlet openings of the hepatic veins. The lumbar veins between the hepatic veins and the renal veins are thus occluded. The interlocking section 114 is preferably positioned or realised such that it leaves the flow path through the hepatic veins substantially undisturbed. The flow path of the normal systemic flow in the inferior vena cava to the right atrium is thus occluded and a selected flow path communicating with the hepatic veins is established through a selectable lumen 106 of the upper or lower catheter stem 104.

FIG. 4b shows schematically another combination of the inventive catheter stents 502,508 similar to that of FIG. 4a. However, in this combination an upper stent 502 in the embodiment with a distal permeable part 102a and a proximal unpermeable part 102b is combined with a lower stent 508 having the whole or the major part 102b of the expandable structure coated to be unpermeable for liquid leaving a shorter permeable part over the hepatic veins.

FIG. 4c shows the embodiment of FIG. 4a applied in a liver perfusion system with an upper catheter 502a and a lower catheter 502b interlocked in the area of the hepatic veins. Each of the two catheters are realising an occlusive seal in the unpermeable proximal part and a flow path through the intermediate permeable parts of the interlocked catheters. However, in embodiments involving the occlusion of vena cava inferior it is required that a flow path for shunting the venous blood flow from inferior vena cava to the right atrium is established. As shown in FIG. 4c such a veno-venous shunt path is establish by means of a shunt catheter 250 having inlet openings 254 and a shunt path lumen 252 with an outlet 256 adapted for connection to an extracorporeal tubing. The shunt path catheter 250 is preferably connected to the previously explained (cf. FIG. 2) second catheter 212 that has its outlet in the upper body half. As for the rest the functionality in the system of FIG. 4c is explained in connection with FIG. 2.

In one embodiment of the liver perfusion system according to FIG. 4c, the flow paths are configured such that there is no flow path through the upper catheter whereas a flow path is arranged through a lumen in the lower catheter and through the hepatic veins. Any pharmaceutical in a perfusion fluid would therefore only or at least substantially only reach the liver.

A variety of the system in FIG. 4c additionally or alternatively comprises an inlet connection 150 to the catheter 240 (above called the fourth catheter) that is coupled to the hepatic artery 290. Infusion of a substance into the hepatic artery is enabled through an infusion conduit 152 and some infusion means 154, e.g. a syringe. Experimental studies have shown that substances, for example cytostatica, i.e. cytotoxic drugs, that are infused via the hepatic artery seem to be absorbed better by the liver tissue than substances infused via veins. In one embodiment the inferior vena cava is therefore occluded e.g. by means of the shown catheter combination (FIG. 4c) however lacking the circulation path through the hepatic artery. An exemplifying perfusion scheme in this configuration would comprise infusing in retrograde a substance such as cytotoxic drugs via the hepatic artery. Then perfusion circulation is performed during e.g. 20 minutes in a circulation flow path through the liver via the portal vein and the hepatic veins, and possibly also via the hepatic artery. In this connection it should be noted that in the embodiment where a perfusion fluid with e.g. cytotoxic drugs is circulated in the perfusion circuit the concentration of a potentially harmful substance should preferably be monitored in order to detect dangerous concentrations or conditions. When instead a premixed and predetermined dose that is known to be safe is infused there is no need for this monitoring.

In connection with the occlusion of the portal vein and the above (FIG. 2) described bypassing of the blood from the lower body half past the liver, there might a be a problem to achieve sufficient bypassing capacity. The amount of the intestinal blood pressing into the portal vein is in a grown up human being in the order of about 700-800 millilitres per minute. The size of the portal vein limits the size of a catheter that is introducible and therefore there is a risk that the lumen in the portal vein bypass catheter is to small to handle the normally required flow rate. A fair judgement concludes that it is probably possible to bypass blood at a flow rate of about 300-400 millilitres/minute from the bowel. Therefore, in one embodiment of the inventive liver perfusion treatment method, the patient is given Vasopresin, or some other generic or similar substance that impedes or restrains haemorrhages. The effect of such a substance is stasis of the blood flow in particular in the intestines, which is acceptable and harmless for a time period in the range of about 30 minutes or so.

Another way of achieving an increased bypass flow from the portal vein is to introduce an alternative or complementary bypass catheter 160 into the portal vein upstream the occlusive seal 236. A lumen 161 having an inlet opening 162 is via an extracorporeal conduit connected to the second catheter 212 (described above) via an inlet connection 164. This catheter can be introduced into the portal vein through a second transhepatic puncture. An alternative is to introduce the bypass catheter 160 into the portal vein via the umbilical vein, that is the vein leading from the navel to the liver and which is normally closed and unused after birth. In some cases it might in fact be impossible to enter the portal vein transhepatically, particularly in the treatment of liver cancer since cancer tumours are hard and can in practical cases fill about 50-70 percent of the cancer diseased liver.

In embodiments of the invention, wherein the bypass catheter 160 is introduced into the portal vein via the umbilical vein, the catheter 160 can be configured to have an occlusive seal or balloon whereby the occlusive catheter 230 and the occlusive seal 236 can be omitted.

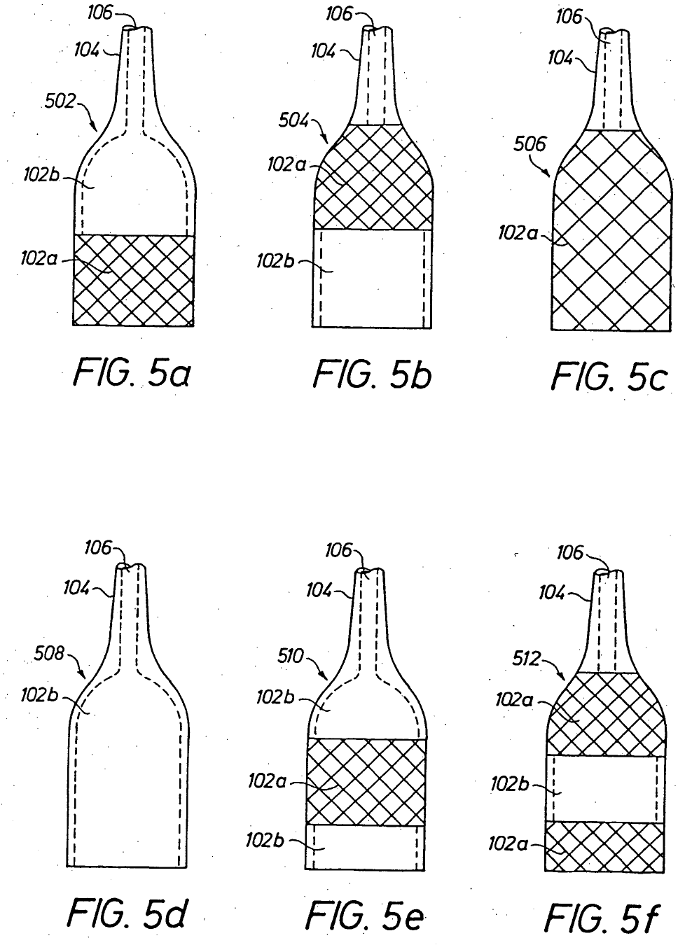

FIG. 5a-5f show different embodiments of the inventive stent 102 with an expandable structure that in its expanded state takes on a funnel like or bell like shape with its distal part taking on a tubular shape of selectable length. A selected first part 102a is permeable for a liquid flow and is preferably made of an expandable mesh structure. A selected second part 102b of the expandable structure is coated with an unpermeable material in order to occlude a flow path from liquid flow. The configuration of length, diameter in unexpanded and expanded state as well as the selection and distribution of permeable and unpermeable part of the stent is dependent on the specific application.

In the embodiment of FIG. 5a the stent 502 comprises an unpermeable part 102b attached to the catheter stem 104 and a permeable part 102a at the distal tip portion. This embodiment enables occlusion of a blood vessel in which the stent is applied (hereinafter called the main vessel), establishing of a flow path to or from the inside of the stent through a lumen 106 and a flow path in branching vessels through the permeable part 102a.

In the embodiment of FIG. 5b the stent 504 comprises a permeable part 102a attached to the catheter stem 104 and an unpermeable part 102b at the distal tip portion. This embodiment enables occlusion of a branching vessel by means of the distal unpermeable part 102b and a maintained flow path in the main vessel through the permeable part 102a.

In the embodiment of FIG. 5c the stent 506 comprises a permeable part 102a over substantially the whole stent structure that is attached to the catheter stem 104. This embodiment enables a maintained flow path in the vessel and is mainly applicable in combination with any of the other embodiments for the purpose of positioning and interlocking.

In the embodiment of FIG. 5d the stent 508 comprises an unpermeable part 102b over substantially the whole stent structure that is attached to the catheter stem 104. This embodiment enables occlusion of the main vessel as well as branching vessels.

In the embodiment of FIG. 5e the stent 510 comprises a first unpermeable part 102b attached to the catheter stem 104, an intermediately positioned permeable part 102a and a distal unpermeable part 102b. This embodiment for example enables occlusion of the main vessel, a flow path through a first branching vessel and occlusion of a second branching vessel situated at a distance from the first branching vessel.

In the embodiment of FIG. 5f the stent 512 comprises a first permeable part 102a attached to the catheter stem 104, an intermediately positioned unpermeable part 102b and a distally positioned second permeable part 102a. This embodiment enables a maintained flow path in the main vessel, occlusion of a first branching vessel and a flow path through a second branching vessel at a distance from the first branching vessel. The distal permeable part may also be used solely for interlocking purposes in combination with any of the other embodiments.

Furthermore, in varieties of the above described embodiments a flow path through an optional lumen 106 can be established for infusion to or evacuation of fluid from the inside of the stent.

The different varieties of the stent are in different embodiments of the invention combined in configurations that are dependent on the specific application. In use such stent combinations are positioned mutually opposing in a blood vessel to interact in interlocking, in the occlusion of a flow path or in the establishing of a flow path, respectively.

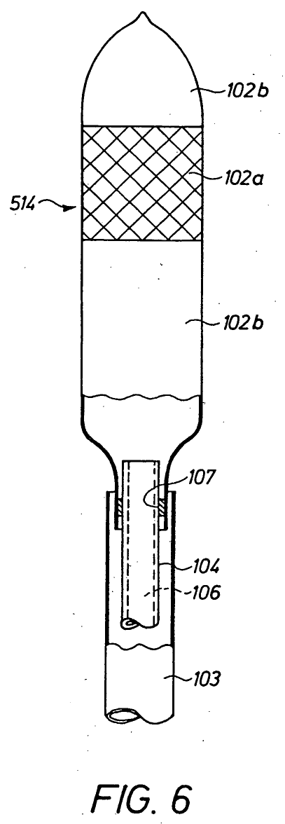

FIG. 6 shows an embodiment of a stent 514 and an infusion catheter having a catheter stem 104 and an inner lumen 106. The catheter is adapted to be introduced by means of an introducer sheath 103, which sheath 103 is retracted when the stent has been delivered into a predetermined position. The stent 514 comprises a distally positioned first unpermeable part 102b, an intermediately positioned permeable part 102a, and a proximally positioned second unpermeable part 102b. Further, when removing the sheath 103 the stent 514 is expanded to an expanded state. In the expanded state, the distal first part 102b of the stent 514 takes on the form of an elongated umbrella and the proximal second part 102b takes on the form of an elongated funnel or an elongated funnel-like structure. Further, the distal part of the first unpermeable part 102b is closed, i.e. there is no opening in the distal part. The distal part of the of the first unpermeable part 102b can for example be tied together. The second unpermeable part 102b is configured to be attached to the catheter stem 104 in the fixation area 107. However, the catheter and the stent can of course also be manufactured as an integrated part.

For the purpose of interlocking in stent combinations, the inventive stent comprises an interlocking mechanism. In a preferred embodiment where the expandable structure is made by means of a possibly partly coated mesh, the interlocking mechanism is inherent in the mesh structure and its functionality. There are also other conceivable alternative or additional interlocking mechanisms, for example hooks or friction enhancing surface structures.

The invention has been described by means of exemplifying embodiments and is defined by the scope of the accompanying claims.

1. A catheter comprising a catheter stem and a radially expandable structure, said expandable structure being configured to adopt in an expanded state a funnel like shape at a first proximal part that is attached to an end of said catheter stem and an elongate substantially tubular shape at a second distal part.

2. The catheter of claim 1, wherein the expandable structure comprises a fluid permeable part.

3. The catheter of claim 1, wherein the expandable structure comprises an unpermeable part that is unpermeable by a fluid.

4. The catheter of claim 1, wherein the expandable structure comprises a fluid permeable part and a fluid unpermeable part.

5. The catheter of claim 1, further comprising an interlocking mechanism configured for interlocking two such catheters being arranged in a mutually opposed position.

6. The catheter of claim 1, wherein the expandable structure is realised by means of a mesh structure or a mesh like wire structure with a radial spring action.

7. The catheter of claim 1, wherein the expandable structure is realised by means of a mesh structure with a radial spring action and the unpermeable part is realised by coating a portion of the mesh structure with a coating material that is unpermeable by a fluid.

8. The catheter of claim 1, wherein the expandable structure is realised by means of a mesh with a radial spring action and comprising an interlocking mechanism configured for interlocking two such catheters being arranged in a mutually opposed position, said interlocking mechanism being realised by means of said spring action of said mesh structure.

9. A catheter set comprising a first and a second catheter according to claim 1 and configured to be arranged mutually opposing in a blood vessel.

10. A catheter comprising:
a radially expandable occlusive seal at a distal portion having an elongate tubular shape, said tubular shaped portion at its envelope surface being coated with a sealing film occluding radial flow; and
a proximal portion being attached to a catheter stem, said proximal portion being permeable for a fluid;
wherein a flow path is enabled axially through the tubular shaped portion and the proximal portion.

11. A catheter comprising:
a radially expandable occlusive seal at a proximal portion being attached to a catheter stem and having a funnel like shape, said funnel shaped portion at its envelope surface being coated with a sealing film occluding axial flow; and
a distal portion having an elongate tubular shape being permeable for a fluid;
wherein a flow path is enabled radially through the envelope of the tubular shaped portion.

12. A catheter comprising:
a radially expandable occlusive seal at a proximal portion being attached to a catheter stem and having a funnel like shape, said funnel shaped portion at its envelope surface being coated with a sealing film occluding axial flow;
an intermediary portion having an elongate tubular shape being permeable for a fluid; and
a radially expandable occlusive seal at a distal portion and having an umbrella like shape, said umbrella shaped portion at its envelope surface being coated with a sealing film occluding flow though said distal portion;
wherein a flow path is enabled radially through the envelope of the tubular shaped portion.

13. The catheter of claim 1, further comprising a lumen in the catheter stem and having an orifice inside the expandable structure to enable a flow path communicating with the inside of the expandable structure.

14. The catheter of claim 1, wherein the catheter is configured to be placed in the inferior vena cava in the area of the hepatic veins.

15. The catheter unit according to claim 1, wherein the catheter is arranged to be used in a liver perfusion system.

16. A perfusion system for non-surgically isolating and perfusing a liver of a living being with a perfusion fluid, comprising:
a catheter set according to claim 1 configured to be positioned in the inferior vena cava in the area of the hepatic veins.

17. The perfusion system of claim 16, wherein the catheter set comprises a catheter having:
a radially expandable occlusive seal at a distal portion having an elongate tubular shape, said tubular shaped portion at its envelope surface being coated with a sealing film occluding radial flow; and
a proximal portion being attached to a catheter stem, said proximal portion being permeable for a fluid;
wherein a flow path is enabled axially through the tubular shaped portion and the proximal portion;
the catheter being configured to be placed in the inferior cava such that the hepatic veins are occluded by the coated tubular shaped portion and such that the main flow in the inferior vena cava is maintained through said axial flow path.

18. The perfusion system of claim 16, wherein the catheter set comprises two catheters having:
a radially expandable occlusive seal at a proximal portion being attached to a catheter stem and having a funnel like shape, said funnel shaped portion at its envelope surface being coated with a sealing film occluding axial flow; and
a distal portion having an elongate tubular shape being permeable for a fluid;
wherein a flow path is enabled radially through the envelope of the tubular shaped portion;
the catheters being configured to be placed mutually opposing in the inferior vena cava such that said inferior vena cava is occluded between the hepatic veins and the entrance into the right atrium and below the hepatic veins by said coated funnel shaped portion and such that a flow path communicating with the hepatic veins is maintained through said radial flow path.

19. The perfusion system of claim 16, wherein the catheter further comprises a catheter having:
a radially expandable occlusive seal at a proximal portion being attached to a catheter stem and having a funnel like shape, said funnel shaped portion at its envelope surface being coated with a sealing film occluding axial flow;
an intermediary portion having an elongate tubular shape being permeable for a fluid; and
a radially expandable occlusive seal at a distal portion and having an umbrella like shape, said umbrella shaped portion at its envelope surface being coated with a sealing film occluding flow though said distal portion;
wherein a flow path is enabled radially through the envelope of the tubular shaped portion;
the catheter being configured to be placed in the inferior cava such that said inferior vena cava is occluded between the hepatic veins and the entrance into the right atrium and below the hepatic veins by said coated umbrella shaped portion and coated funnel shaped portion and such that a flow path communicating with the hepatic veins is maintained through said radial flow path.

20. A medical device for controlling the flow path in the inferior vena cava comprising a catheter according to claim 1.
